(19) United States
(12) Patent Application Publication
(10) Pub. No.: US 2004/0193119 A1
(43) Pub. Date: Sep. 30, 2004

(54) Catheter port assembly for extracorporeal treatment

(76) Inventors: Bernard Canaud, Montpellier (FR); Kenneth J. Chesnin, Philadelphia, PA (US)

Correspondence Address: MONTE & MCGRAW, PC, 4092 SKIPPACK PIKE, P.O. BOX 650, SKIPPACK, PA, 19474, US

(21) Appl. No.: 10/778,660

(22) Filed: Feb. 13, 2004

Related U.S. Application Data
(60) Provisional application No. 60/447,080, filed on Feb. 13, 2003.
(60) Provisional application No. 60/494,894, filed on Aug. 13, 2003.

(51) Int. Cl. A61M 5/00

(52) U.S. Cl. 604/247

(57) ABSTRACT

A catheter port assembly is disclosed. The assembly includes an adapter having a body having a proximal end, a distal end, and a passageway extending therethrough between the proximal end and the distal end. The distal end is adapted to engage a catheter. The assembly further includes a tunneler releasably connectable to the proximal end of the adapter and a catheter port, having a valve disposed therein, releasably connectable to the proximal end of the adapter. In an alternative preferred embodiment, the valve may be disposed inside of the adapter, rather than the catheter port. Alternatively, the body of the port assembly may be one piece, having a valve disposed therein. A method of installing and operating the catheter port assembly is also provided.

### CROSS-REFERENCE TO RELATED APPLICATIONS

This patent application claims the benefit of U.S. Provisional Patent Application Serial No. 60/447,080, filed on Feb. 13, 2003 and U.S. Provisional Patent Application Serial No. 60/494,894, filed on Aug. 13, 2003.

### FIELD OF THE INVENTION

The present invention relates to a catheter port assembly and a method of inserting the catheter port assembly.

### BACKGROUND OF THE INVENTION

Catheters for extracorporeal blood purification may be located in various venous locations and cavities throughout the body of a patient for administration of solutes and for removal of toxins and fluids from the body via an extracorporeal blood circulation. Such venous catheterization may be performed by using a single catheter having multiple lumens. A typical example of a multiple lumen catheter is a dual lumen catheter in which one lumen serves to aspirate blood (arterial line) and the other lumen serves to restitute cleaned blood (venous line). An example of such a dual lumen catheter assembly is the SPLIT CATH® catheter, manufactured by Medical Components, Inc. of Harleysville, Pa. Catheterization may also be performed by using separate, single lumen catheters inserted through the same incision into the deep vein to be catheterized. Such dual catheter assemblies are also manufactured by Medical Components, Inc. of Harleysville, Pa.

Generally, to insert any catheter into a deep vein or other blood vessel, the vessel is identified by aspiration with a long hollow needle in accordance with the well known Seldinger technique. When blood enters a syringe attached to the needle, indicating that the vessel has been found, a thin guide wire is then introduced, typically through a syringe needle or other introducer device into the interior of the vessel. The introducer device is then removed, leaving the distal end portion of the guide wire that has been inserted into the vessel within the vessel and the opposing proximal end of the guide wire projecting beyond the surface of the skin of the patient. At this point, several options are available to a physician for catheter placement. The simplest option is to pass a semi-rigid catheter into the vessel directly over the guide wire. The guide wire is then removed, leaving the catheter in position within the vessel. If the catheter to be inserted is significantly larger than the guide wire or is constructed from soft, flexible polymer material, a vein dilator device in a sheath is passed over the guide wire to enlarge the hole and to facilitate the introduction of the catheter. The guidewire and dilator are removed and the catheter is inserted through the sheath, into the vein. The sheath is removed, leaving the catheter in place.

For chronic catheterization, in which the catheter is intended to remain inside the patient for an extended period of time, such as for weeks or even months, it is typically desired to subcutaneously tunnel the catheter into the patient using various tunneling techniques. The proximal end of the catheter is typically tunneled after the catheter is inserted into the patient's vein. The subcutaneous tunnel provides a stable anchor to prevent the proximal end of the catheter from moving and possibly becoming dislodged, which could result in patient discomfort and risk of injury, such as infection, inflammation, or accidental withdrawal.

U.S. Pat. No. 4,431,426 to Groshong et al. discloses an apparatus for forming a subcutaneous tunnel during catheter insertion. The apparatus includes a coupler that includes a distal end connected to a catheter and a threaded proximal end. The proximal end of the coupler is threadably connected to a passer having a tip with a sharp forward end. The passer is guided into and under the skin of the patient in the fat tissue, forming a subcutaneous tunnel. After the tunnel is formed to a desired length, the passer is guided through and exterior to the skin. The tip is unthreaded from the coupler and a flow reducing adapter is threaded onto the coupler. A fitting or a cap may be alternatively connected to a proximal end of the flow reducing adaptor. However, when the catheter is being connected to or disconnected from a hemodialysis machine, during the time period when the flow reducing valve is not connected to anything, fluid, such as catheter lock solution and/or blood from the patient, may flow from the catheter, potentially contaminating equipment or medical personnel in the area. Further, during the time period when the flow reducing valve is open, air may be aspirated into the catheter due to negative intrathoracic pressure, creating a potential air embolism.

It would be beneficial to provide a catheter port assembly in which a tunneler and port are alternatively releasably connectable to a catheter through an adapter, and in which a valve is disposed within the port to allow fluid flow in a first direction but to restrict fluid flow in a second direction such that, when the port is open, fluid does not flow from the catheter and air aspiration is not possible.

### BRIEF SUMMARY OF THE INVENTION

Briefly, the present invention provides a catheter port assembly comprising a body having a proximal end, a distal end, and a passageway extending therethrough between the proximal end and the distal end. The distal end is adapted to engage a catheter. The assembly further comprises a tunneler releasably connectable to the proximal end of the body and a catheter port alternatively releasably connectable to the proximal end of the body. A valve is disposed within the catheter port.

The present invention further provides a catheter port assembly comprising a body having a proximal end, a distal end, and a passageway extending therethrough between the proximal end and the distal end. The distal end is adapted to engage a catheter. The assembly further comprises a tunneler releasably connectable to the proximal end of the body and a catheter port alternatively releasably connectable to the proximal end of the body. A valve is disposed within the body.

The present invention further provides a catheter port assembly comprising a body having an outer surface, a proximal end, a distal end, and a passageway extending between the distal and proximal ends. There is, disposed along the passageway between the distal and proximal ends, a valve. The distal end is adapted to engage a catheter. The catheter port assembly further comprises a tunneler releasably connectable to the proximal end of the body.

Also, the present invention provides a method of subcutaneously securing a catheter to a patient comprising: providing a catheter port assembly comprising a body having a proximal end, a distal end, and a passageway extending therethrough between the proximal end and the distal end. The distal end is connected to a catheter lumen and the proximal end is releasably connected to a tunneler. The method further comprises using the tunneler to form a subcutaneous tunnel in a patient; advancing the tunneler and at least a portion of the body through the tunnel and out of the tunnel; removing the tunneler from the body; and releasably connecting a distal end of a catheter port to the proximal end of the body, wherein the catheter port comprises a valve disposed therein.

Also, the present invention provides a method of subcutaneously securing a catheter to a patient comprising: providing a catheter port assembly comprising a body having a proximal end, a distal end, and a passageway extending therethrough between the proximal end and the distal end. The distal end of the body is connected to a catheter lumen and the proximal end of the body is releasably connected to a tunneler. The method further comprises using the tunneler to form a subcutaneous tunnel in a patient; advancing the tunneler and at least a portion of the body through the tunnel and out of the tunnel; removing the tunneler from the body; and releasably connecting a distal end of a catheter port to the proximal end of the body, wherein the body comprises a valve disposed therein.

The present invention further provides a method of subcutaneously securing a catheter to a patient comprising: providing a catheter port assembly having a proximal end, a distal end, a passageway extending therethrough between the proximal end and the distal end, a valve disposed along the passageway between the proximal and distal ends and a tunneler releasably connected to the proximal end. The method further provides using the tunneler to form a subcutaneous tunnel in a patient and advancing the tunneler and at least a portion of the catheter port assembly through the tunnel and out of the tunnel. The method further provides removing the tunneler from the proximal end of the catheter port assembly.

### BRIEF DESCRIPTION OF THE DRAWINGS

The accompanying drawings, which are incorporated herein and constitute part of this specification, illustrate the presently preferred embodiment of the invention, and, together with the general description given above and the detailed description given below, serve to explain the features of the invention. In the drawings:

FIG. 1 is an exploded side view of the catheter port assembly according to a first embodiment of the present invention.

FIG. 2 is a sectional view of an adapter of the assembly shown in FIG. 1, taken along line 2--2 of FIG. 1.

FIG. 3 is an alternate embodiment of the adapter of the assembly shown in FIG. 2.

FIG. 4 is an alternate two-piece embodiment of the tunneler of the assembly shown in FIG. 1.

FIG. 5 is a longitudinal cross-sectional view of the tunneler shown in FIG. 4.

FIG. 6 is an alternate embodiment of the tunneler of the assembly shown in FIG. 1.

FIG. 7 is a sectional view of the catheter port assembly shown in FIG. 1, taken along the line 7-7 of FIG. 1.

FIG. 8 is an alternate embodiment of the catheter port of the assembly shown in FIG. 7.

FIG. 9 is a side view of the adapter of FIG. 1 being connected to the tunneler of FIG. 1.

FIG. 10 is a side view of the adapter of FIG. 1 having been connected the tunneler of FIG. 1.

FIG. 11 is a side view of the catheter port assembly of FIG. 10 being drawn through a subcutaneous tunnel.

FIG. 12 is a side view of the assembly of FIG. 11, with the tunneler removed, being connected to the port of FIGS. 1 and 2.

FIG. 13 is a perspective view of the port of FIGS. 1 and 7 having been connected to the adapter of FIGS. 1 and 2.

FIG. 14 is a perspective view of a pair of the inventive assemblies connected to a hemodialysis machine.

FIG. 15 is a top plan view of a pair of catheter ports of the invention disposed within a retainer.

FIG. 16 is a side view of the pair of catheter ports of the invention disposed in the retainer as seen along line 16--16 of FIG. 15.

FIG. 17 is a top-plan view of the pair of inventive assemblies being connected to a lock.

FIG. 18 is a top-plan view, partially torn away, of the pair of inventive assemblies inserted into the lock of FIG. 17.

FIG. 19 is a side view of a port assembly.

FIG. 20 is a sectional view of the port assembly of FIG. 19 taken along line 20-20.

FIG. 21 is a flow chart describing a method of using a catheter tunneler adapter.

FIG. 22 is a perspective view of a pair of inventive assemblies disposed in a snap retainer.

### DETAILED DESCRIPTION OF THE INVENTION

In the drawings, like numerals indicate like elements throughout. Certain terminology is used herein for convenience only and is not to be taken as a limitation on the present invention. The words “proximal” and “distal” refer to directions away from and closer to, respectively, the insertion tip of the catheter in a catheter assembly utilizing a catheter port adapter assembly 100 according to the present invention. The terminology includes the words above specifically mentioned, derivatives thereof, and words of similar import. The following describes preferred embodiments of the invention. However, it should be understood, based on this disclosure, that the invention is not limited by the preferred embodiments described herein.

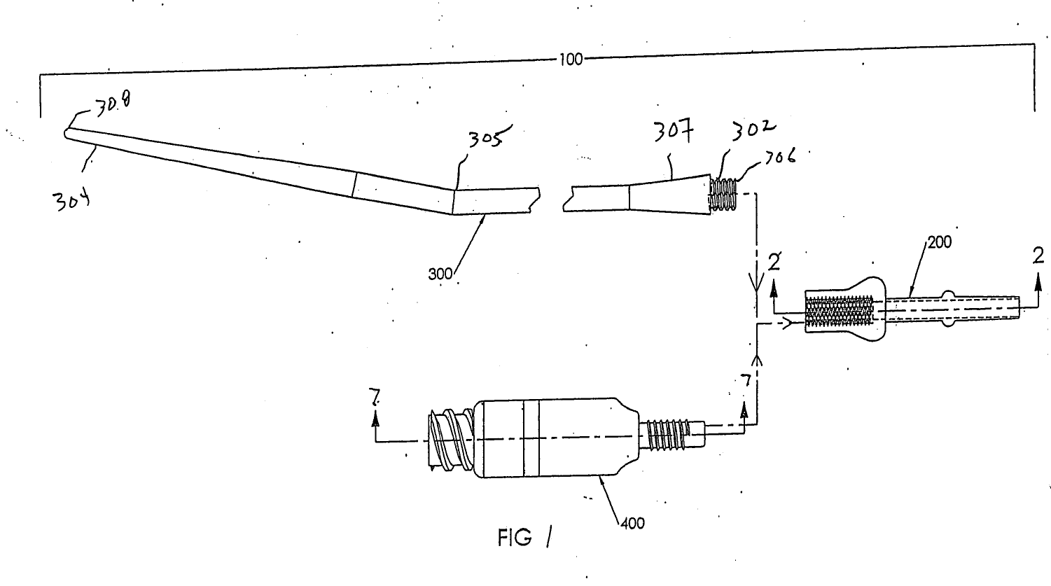

Referring now to FIG. 1, a catheter port adapter assembly 100 according to an embodiment of the present invention is shown. The assembly 100 is used to facilitate subcutaneous tunneling of a catheter in a patient to retain the catheter in the patient for chronic medical procedures, such as hemodialysis. The assembly 100 is also used to connect the catheter to a medical device, such as a hemodialysis machine. The assembly 100 includes an adapter 200, a tunneler 300, and a port 400. The adapter 200 is alternately releasably connectable to the tunneler 300, to facilitate the tunneling, and to the port 400, to connect the catheter to the medical device.

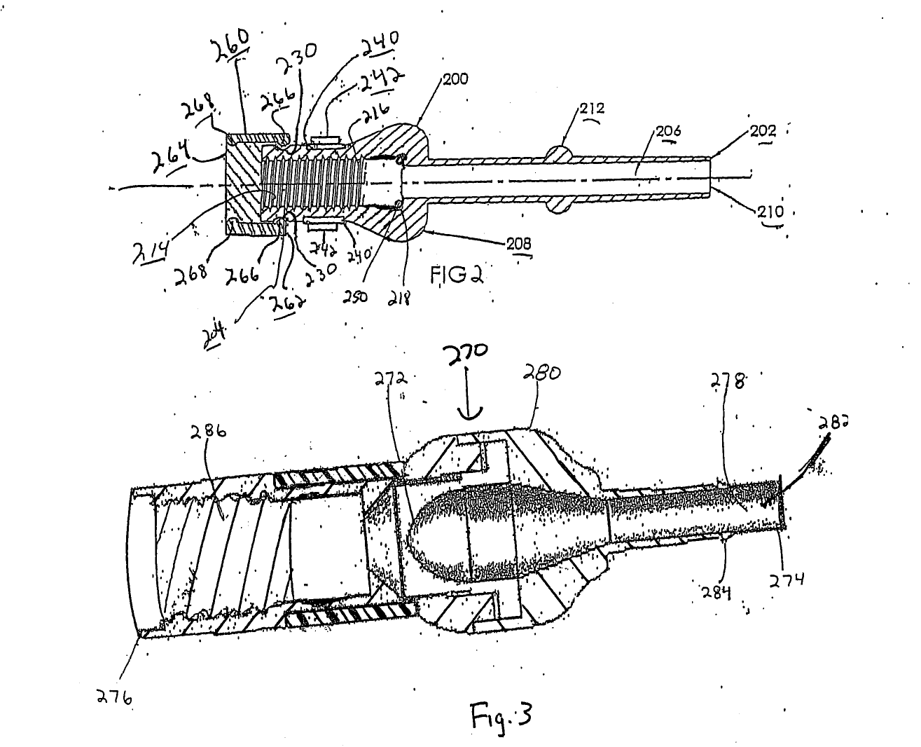

Referring now to FIG. 2, the adapter 200 is generally cylindrical in shape and includes a distal end 202, a proximal end 204, and a longitudinal passageway 206 extending therethrough between the distal end 202 and the proximal end 204. Preferably, the passageway 206 has a generally constant diameter between the distal end 202 and the proximal end 204 to promote laminar fluid flow through the passageway 206. A generally bulbous portion 208 is disposed along the adapter 200 between the distal end 202 and the proximal end 204. The bulbous portion 208 may be knurled or otherwise roughened to facilitate fibrous scarring and subcutaneous anchoring after the adapter 200 is inserted into a subcutaneous tunnel, as is disclosed later herein. The distal end 202 of the adapter 200 tapers from narrower to wider from a distal tip 210 to the bulbous portion 208. A retaining nub 212 is disposed along the distal end 202 between the distal tip 210 and the bulbous portion 208. Alternatively, a barb (not shown) may be used in place of the retaining nub 212. The proximal end 204 of the adapter 200 preferably includes a threaded connection 214 for connecting the tunneler 300 and the port 400 to the adapter 200. There may be an O-ring 250 inserted into the passageway 206 to help facilitate sealing between the adapter 200 and the port 400 or the tunneler 300. The O-ring may be merely inserted into the passageway 206 of the adapter 200, or the O-ring 250 may be set inside of a circumferential reveal 218 located on the inner surface of the passageway 206. While a threaded connection 214 is shown in FIG. 2 having female threads, those skilled in the art will recognize that the adapter 200 may alternatively have male threads (not shown). Alternatively, other connection methods, known to those skilled in the art, may be used to releasably connect the adapter 200 to the port 400 or the tunneler 300.

Referring still to FIG. 2, a ring 240, preferably constructed of silicone, polyurethane or some other suitable biocompatible material disposed around the port 200 between the proximal end 204 and the generally bulbous portion 208. The ring 240 aids in the healing process by improving tissue in-growth. A fabric ingrowth cuff 242 may also be fixedly attached to the ring 240 to further aid in tissue in-growth and fixation.

There may also be a recess 230 notched circumferentially around the outside of the port between the ring 240 and the proximal end 204. A snap ring 260 may be snapped into the recess 230. The snap ring 260 comprises a distal ring end 262 and a proximal ring end 264, wherein the distal ring end 262 has a distal ridge 266 and the proximal ring end 264 has a proximal ridge 268. The snap ring 260 has a generally annular shape and the distal ridge 266 is sized to be disposed within the recess 230 located on the proximal end 204 of the adapter 200. The snap ring 260 is releasably locked around the proximal end 204 of the adapter 200 when the distal ridge 266 is disposed in the recess 230. The snap ring 260 is attached to help ensure that the adapter 200 will not become disengaged from the port 400.

Alternatively, as shown in FIG. 3, an alternate preferred embodiment of an adapter 270 may include a valve 272. The adapter 270 is generally cylindrical in shape and preferably includes any of the aforementioned features of the adapter 200 shown in FIG. 2.

Referring still to FIG. 3, the adapter 270 includes a distal end 274, a proximal end 276, and a longitudinal passageway 278 extending therethrough between the distal end 274 and the proximal end 276. The valve 272 is disposed along the longitudinal passageway 278 between the distal end 274 and the proximal end 276. Preferably, the valve 272 allows fluid flow easily in a first direction but restricts fluid flow in a second direction. A preferred valve is a bi-directional valve or a duckbill valve, although those skilled in the art will recognize that other types of valves may be used. A generally bulbous portion 280 is disposed along the adapter 270 between the distal end 274 and the proximal end 276. The bulbous portion 280 may be knurled or otherwise roughened to facilitate fibrous scarring and subcutaneous anchoring after the adapter 270 is inserted into a subcutaneous tunnel, as is disclosed later herein. The distal end 274 tapers from narrower to wider from a distal tip 282 to the bulbous portion 280. A retaining nub 284 is disposed along the distal end 274 between the distal tip 282 and the bulbous portion 280. The proximal end 276 of the adapter 270 includes a threaded connection 286 disposed along the longitudinal passageway 278. Although FIG. 3 shows an embodiment of an adapter 270 having female threads 286, those skilled in the art will recognize that the adapter 270 may alternatively have male threads (not shown) or some other method of connecting to the port or tunneler.

Referring back to FIG. 1, the tunneler 300 is generally elongated with a circular cross-section and includes a distal end 302 and a proximal end 304. Preferably, between the distal end 302 and the proximal end 304, the tunneler 300 includes a bend 305 for facilitating tunneling during catheter insertion. Alternatively, the tunneler 300 may be straight, having no bend between the distal end 302 and the proximal end 304. In one preferred embodiment, the distal end 302 includes a threaded connection 306 that is adapted to threadably connect to the threaded connection 214 in the adapter 200. While FIG. 1 shows male threaded connection 306 on the tunneler 300, those skilled in the art will recognize that the threaded connection 306 on the tunneler 300 may also be female to mate with an alternative male thread in the adapter 200, as previously discussed. Those skilled in the art will also recognize that other known methods may be used to releasably connect the tunneler 300 to the adapter 200.

The distal end 302 of the tunneler 300 further includes a tapered portion 307 that tapers from wider to narrower from the threaded connection 306 toward the bend 305. The widest end of the tapered portion 307 is sized to be approximately the same diameter as the proximal end 204 of the adapter 200 to facilitate a smooth transition between the tunneler 300 and the adapter 200. The proximal end 304 of the tunneler 300 includes a generally blunt tip 308 for tunneling under a patient's skin during catheter insertion. However, those skilled in the art will recognize that the tip 308 may also be sharp in order to allow the tunneler 300 to pierce through the patient's skin during tunneling. The proximal end 304 of the tunneler 300 preferably includes a taper that tapers from narrower to wider from the tip 308 to the bend 305.

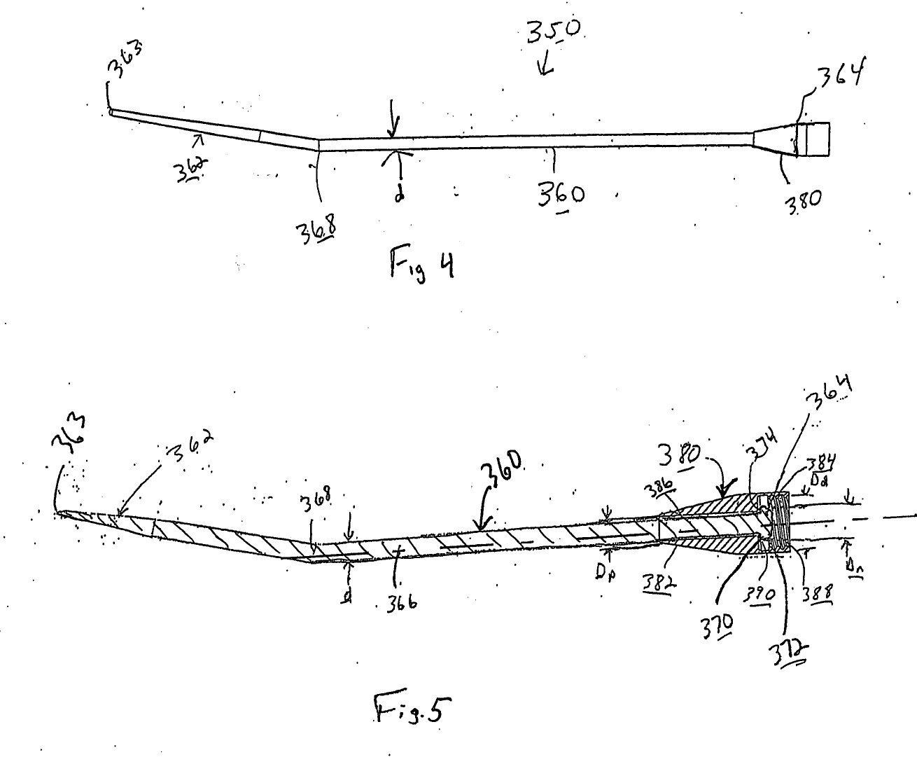

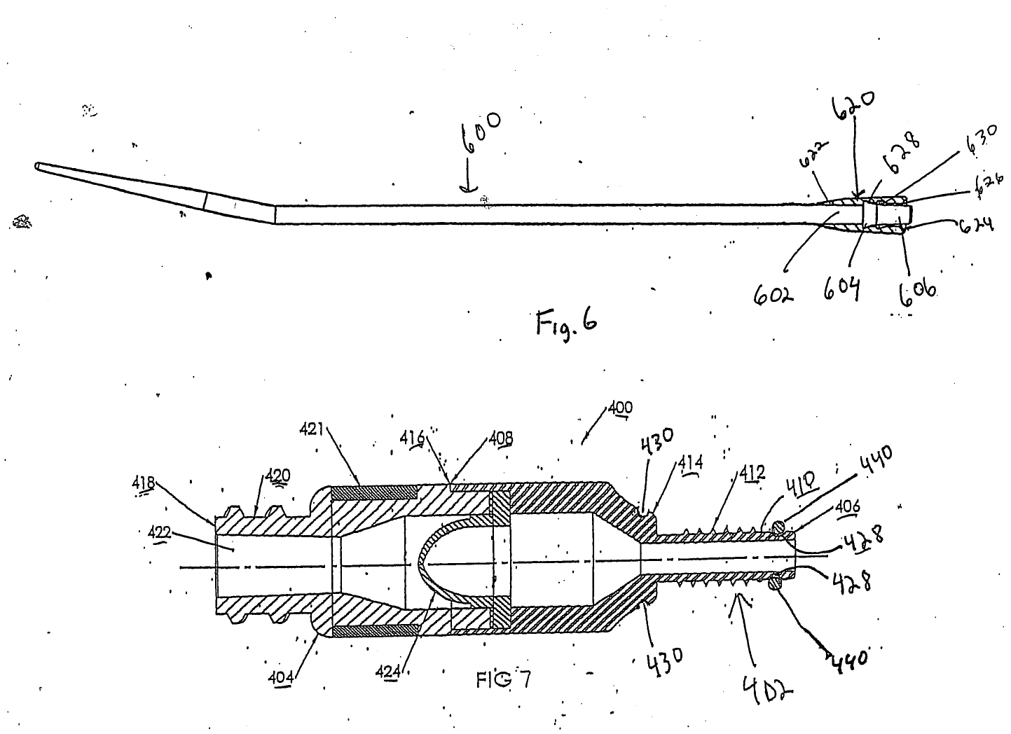

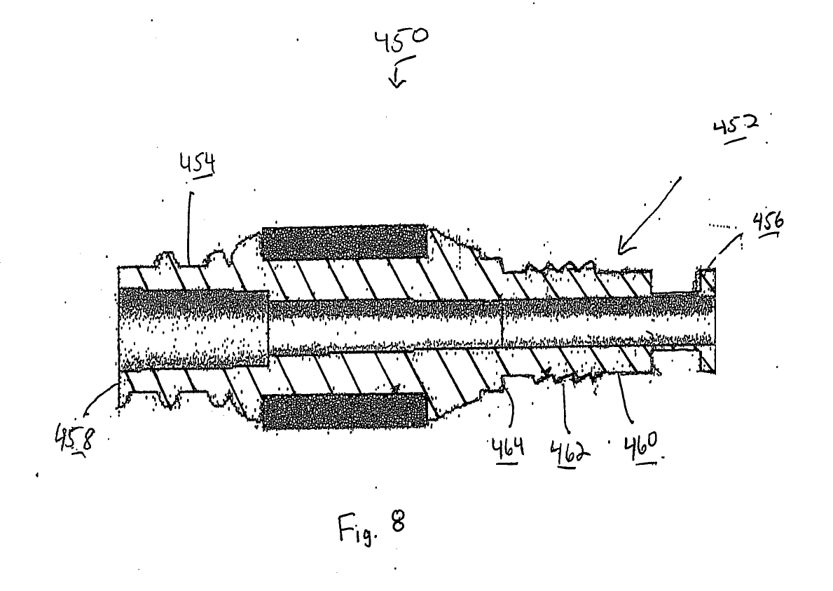

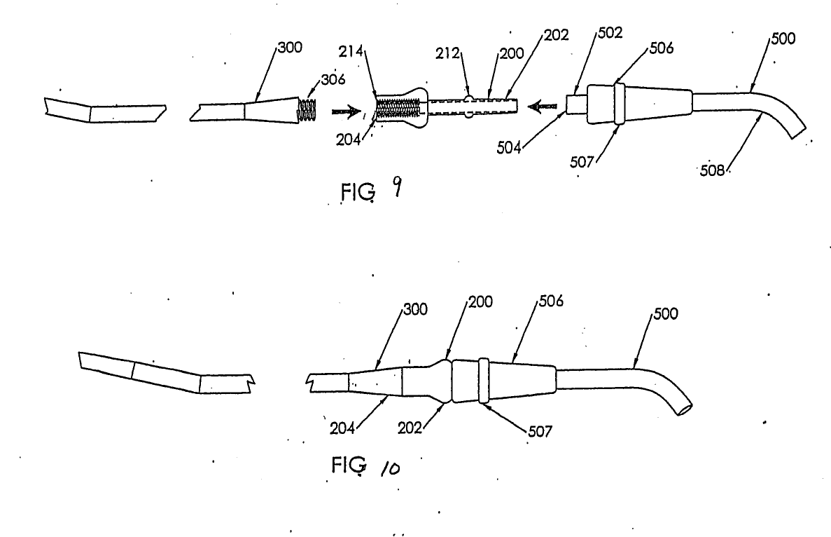

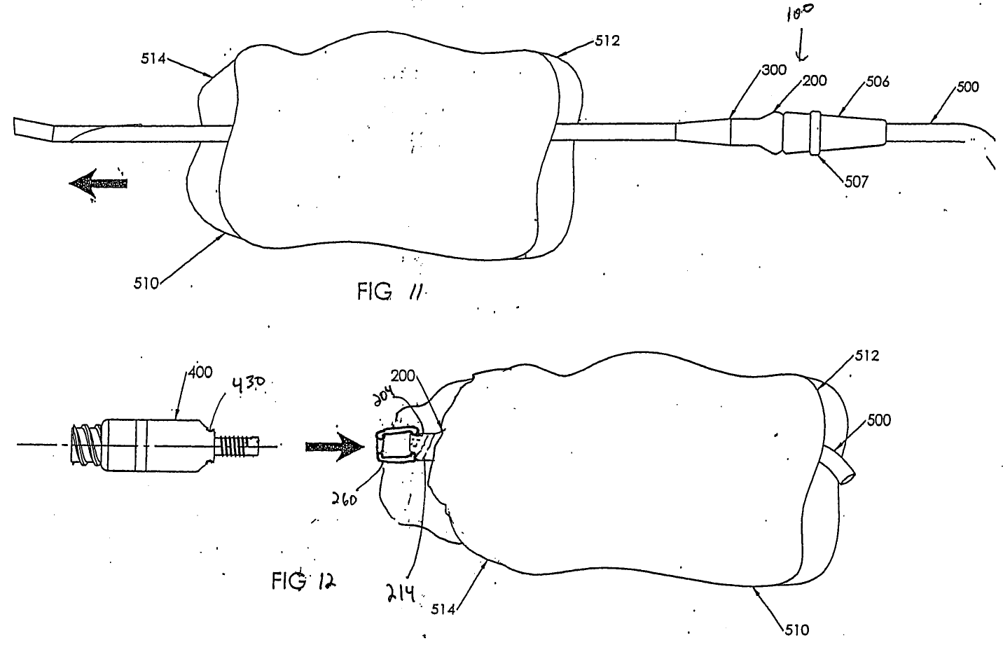

As shown in FIGS. 4 and 5, an alternate embodiment of a tunneler 350 may also be made using a two piece construction. In such a configuration, the tunneler 350 comprises an elongated shaft 360 and a connector piece 380. The elongated shaft 360 comprises a proximal end 362, a distal end 364 and a longitudinal axis 366 extending therethrough between the proximal end 362 and the distal end 364. A bend 368 in the elongated shaft 366 may be located between the distal end 364 and the proximal end 362. Alternatively, the elongated shaft 366 may be straight. The proximal end 362 has a proximal tip 363. At the proximal tip 363, the diameter of the elongated shaft 360 reduces from the original diameter “d” of the elongated shaft 360 down to a point 363. A retaining nub 370 is located at the distal end 364 of the elongated shaft 360. The retaining nub 370 has a larger diameter “Dₙ” than the diameter “d” of the elongated shaft 360. The retaining nub 370 has a retaining shoulder 374 located at the proximal end of the retaining nub 370. The retaining nub 370 may also have tapered sides 372 to allow the longitudinal passageway 206 of the adapter 200 to frictionally engage the sides of the retaining nub 320 in a luer type connection when the adapter 200 is releasably connected to the tunneler 350.

The connector piece 380 comprises a proximal end 382, a distal end 384 and a hollow passageway 386 extending therethrough. The hollow passageway 386 has two different diameters; the hollow passageway 386 has a larger distal diameter, “D_{d}” at the distal end 364 and a smaller proximal diameter, “Dₚ,” at the proximal end 382. The proximal diameter “Dₚ” of the hollow passageway 386 is sized so that the connector piece 380 may be slid over the elongated shaft 360 from the proximal end 362. The hollow passageway 386 is larger in diameter at the distal end 384 of the connector piece 380. Threads 388 are disposed at the distal end 384 of the hollow passageway 386 to releasably engage the proximal end 204 of the adapter 200. Proximally of the threads 388, along the hollow passageway 386, is a transition shoulder 390 where the diameter of the hollow passageway 386 reduces from the larger distal diameter “D_{d},” that is sized to accept the proximal end of the adapter 200, to the smaller proximal diameter “Dₚ” that is sized to merely slip over the elongated shaft 360. The connector piece 380 is therefore free to rotate about the elongated shaft 360. This rotation about the elongated shaft 360 will allow the adapter 200 to be releasably connected to the tunneler 350 by merely engaging the threads 214 of the adapter 200 to the threads 388 of the connector piece 380 and rotating the connector piece 380 about the elongated shaft 360 until the adapter 200 is secured to the tunneler 350. The smaller proximal diameter “Dₚ” is marginally larger than the diameter of the elongated shaft 360; while the larger distal diameter “D_{d}” is larger than the diameter “Dₙ.” This configuration allows the connector piece 380 to be slid distally over the proximal end 362 of the elongated shaft 360 until the transition shoulder 390 of the connector piece 380 engages the retaining shoulder 374. Preferably when the adapter 200 is secured to the tunneler 350, the inside of the proximal end 204 of the adapter 200 is pressed around and against the tapered sides 372 of the retaining nub 370. It is also preferable, when using the two-piece tunneler 350, that the proximal end 204 of the adapter 200 is pressed against and around the tapered sides 372 of the retaining nub 370

While it is preferable for the adapter 200 to be releasably connected to the tunneler 350 by a threaded connection, it will be known to those skilled in the art that any method of releasably connecting the adapter 200 to the tunneler 350 may be used. As shown in FIG. 6, an alternate embodiment of an adapter 600 may have compression sleeve 620 that stretches over a distal end 602 of the tunneler 600 to hold the adapter 200 and the tunneler 600 together. The compression sleeve is generally shaped like the connector piece 380 described above. The compression sleeve 620 has a proximal end 622 and a distal end 624 and a longitudinal passageway 626 extending therethrough. A proximal recess 628 and a distal recess 630 are circumferentially disposed on the longitudinal passageway 626 between the distal end 624 and the proximal end 622 of the compression sleeve 620. The distal end 602 of the tunneler 600 has a retaining ridge 604 and a tapered luer tip 606. The compression sleeve 620 is disposed about the tunneler 600 so that the retaining ridge 604 is frictionally disposed within the proximal recess 628 of the compression sleeve 620. Preferably the tension of the compression sleeve 620, just as the threads in the threaded connection, will pull the tunneler 600 distally towards the adapter 200 so that the tapered luer tip 606 will be frictionally disposed within the proximal end 204 of the adapter 200 to create a seal and restrict fluid flow through the adapter 200 during tunneling. It will be known to those skilled in the art that where a tunneler 600 and a compression sleeve 620 are used, the adapter 200, and correspondingly, the port 400 will be constructed to fit into the compression sleeve 620. The adapter 200 will be constructed so that the proximal end 204 of the adapter forms a seal with the tapered luer tip 624 of the tunneler 600 as well as the tunneler 400.

Referring now to FIG. 7, the port 400 includes a distal portion 402 and a proximal portion 404. The distal portion 402 includes a distal end 406 and a proximal end 408. The distal end 406 includes a generally tubular insert 410 that is sized to be inserted into the passageway 206 at the proximal end of 204 of the adapter 200. The insert 410 includes a threaded connection 412 that is adapted to threadably connect to the threaded connection 214 in the adapter 200. A shoulder 414 is disposed proximal of the threaded connection 412 to engage the proximal end 204 of the adapter 200 when the port 400 is connected to the adapter 200.

The proximal portion 404 includes a distal end 416 and a proximal end 418. The proximal end 408 of the distal portion 402 mates with the distal end 416 of the proximal portion 404. The proximal end 418 of the proximal portion 404 includes a threaded luer connector 420 for connection to an external device, such as a hemodialysis machine.

The port 400 includes a passageway 422 extending therethrough from the distal end 406 of the distal portion 402 to the proximal end 418 of the proximal portion 404. A valve 424 is disposed within the passageway 422 generally at the junction of the proximal end 408 of the distal portion 402 and the distal end 416 of the proximal portion 404. Preferably, the valve 424 allows fluid flow easily in a first direction but restricts fluid flow in a second direction. It should be known that the valve 424 is bidirectional. The valve 424 allows flow in both directions but restricts flow in a non-preferred direction. The valve 424 may be disposed in the port 400 to allow flow in either direction. This is particularly useful in applications such as hemodialysis wherein two separate catheters, or a multiple catheter assembly utilizes two separate valves 424 and the two valves 424 are disposed to allow flow in opposite directions. A preferred valve is a bi-directional valve or a duckbill valve, although those skilled in the art will recognize that other types of valves may be used. Alternatively, the valve 424 could restrict fluid flow in both directions. Preferably, during manufacture of the port 400, the valve 424 is inserted into the passageway 422 prior to connecting the distal portion 402 and the proximal portion 404. An exterior surface of the port 400 between the shoulder 414 and the luer connector 420 may be ridged or knurled to provide a gripping surface to facilitate engagement or disengagement of the port 400 with the adapter 200. Located on the exterior surface of the proximal portion 404 of the port 400 may be a sleeve 421 to help facilitate gripping of the port during operation. The sleeve 421 may be rubber or some other material that facilitates gripping.

Referring now to FIG. 8, an alternate embodiment of a port 450 includes a distal portion 452 and a proximal portion 454. The distal portion 452 includes a distal end 456 and the proximal portion 454 includes a proximal end 458. The distal end 456 includes a generally tubular insert 460 that is sized to be inserted into the proximal end 276 of the adapter passageway 278 of the adapter shown in FIG. 3. A proximal portion of the insert 460 includes a threaded connection 462 that is adapted to threadably connect to the threaded connection 286 in the adapter 270 shown in FIG. 3. Referring to both FIG. 3 and FIG. 8, a shoulder 464 of the port 450 is disposed proximal of the threaded connection 462 to engage the proximal end 276 of the adapter 270 when the port 450 is connected to the adapter 270. Preferably, when the embodiment of the port 450 is used, the port 450 is used in conjunction with the adapter 270 shown in FIG. 3.

Likewise, the port 400 may also be held in place using a compression sleeve 620. While an embodiment of the port 400 adapted to be releasably connected to the adapter 200 using a compression sleeve 620 is not shown, it will be well known to those skilled in the art that in such an embodiment, the distal end of the port 400 will have similar features to the distal end 602 of the tunneler 600 shown in FIG. 6. Correspondingly, the adapter 200 will have features to releasably connect to the port 400 and the tunneler as described previously herein.

Referring now to FIG. 7, an O-ring 440 may be disposed around the tubular insert 410 of the port 400 to ensure a leak proof seal between the port 400 and the adapter 200. The O-ring 440 is disposed in a recess 428 notched circumferentially around the outer surface of the tubular insert 410 of the port 400. When the port 400 and the adapter 200 are releasably connected, the O-ring 440 disposed in the recess 428 of the tubular insert 410 of the port 400 forms a seal against the inner surface of the proximal portion 204 of the adapter 200. Alternatively, in a embodiment including a port 400 having female threads and an adapter having male threads, the O-ring would be disposed around the adapter in a recess similar to the recess 428 shown on the port 400 in FIG. 7. Similarly, in an embodiment wherein the adapter 200 has male threads, there would be a circumferential reveal along the inner surface of the distal portion 402 of the port 400 having female threads.

Referring to FIGS. 2 and 7, the port 400 has a recess 430 located just proximally of the shoulder 414. The proximal ridge 268 of the snap ring 260 is sized to be disposed within the recess 430 of the port 400. Preferably, the proximal ridge 268 of the snap ring 260 is snapped into the ridge 430 of the port 400 prior to releasably connecting the port 400 and the adapter 200. The tubular insert 410 of the port 400 is then threaded into the threaded connection 214 of the adapter 200 and at about the same time that the tubular insert 410 is completely threaded into the threaded connection 214, the distal ridge 266 snaps into the recess 230 on the adapter 200. Preferably, the shoulder 414 of the port 400 engages the proximal end 204 of the adapter 200 at about the same time that the distal ridge 266 snaps into the recess 230 on the adapter 200. Alternatively, in an embodiment, not shown, having an adapter with male threads and a port with female threads, the snap ring 260 would be initially disposed in a recess on the port and would snap into a recess on the port as the adapter was threadably connected to the port.

Preferably, all of the adapter 200, the tunneler 300, and the port 400 are constructed from a bio-compatible non-oxidizing metal, such as stainless steel or titanium, although those skilled in the art will recognize that other bio-compatible materials, including polymers, may be used. The valve 424 is preferably constructed from silicone or some other biocompatible material known to those skilled in the art.

Insertion and operation of the assembly 100 is illustrated in FIGS. 9 through 18. Referring to FIG. 9, the threaded connection 306 of the tunneler 300 is threaded onto the threaded connection 214 of the adapter 200. Although FIG. 9 shows an adapter 200 having female threads and a tunneler 300 having male threads, it will be known to those skilled in the art that the adapter 200 may have male threads and the tunneler 300 may have corresponding female threads, or that some other method of connection may be used to releasably connect the tunneler 300 to the adapter 200.

Referring to FIGS. 9 and 10, the distal end 202 of the adapter 200 is inserted into the proximal end 502 of a lumen 504 of a catheter 500. The distal end 202 of the adapter 200 is inserted sufficiently into the lumen 504 such that the catheter 500 extends over the retaining nub 212. The catheter 500 engages the distal end 202 in a generally leak proof fit. Optionally, an elastic retaining sleeve 506 is disposed over the catheter 500 and the distal end 202 of the adapter 200 to further compress the catheter 500 onto the distal end 202 of the adapter 200. The retaining sleeve 506 is preferably constructed from silicone or some other suitable biocompatible material known to those skilled in the art. Also optionally, a fabric ingrowth cuff 507 may be disposed about at least a portion of the exterior of the retaining sleeve 506 to facilitate securing the catheter 500 to the patient after the catheter 500 is tunneled. As seen in FIGS. 9 and 10, the retaining sleeve 506 is preferably tapered, although those skilled in the art-will recognize that the retaining sleeve 506 need not be tapered. A distal end 508 of the catheter 500 is inserted into the patient by methods well known in the art. The adapter 200, with the tunneler 300 and the catheter 500 connected to the proximal and distal ends 204, 202 of the adapter 200, respectively, is shown in FIG. 10.

FIG. 11 illustrates the tunneler 300 being used to form a subcutaneous tunnel 510 in a patient. Preferably, the distal end of the catheter 500 has already been inserted into the patient according to known techniques. Optionally, the inserting physician may make an incision at what will be a proximal end 514 of the tunnel 510. The inserting physician may also form the tunnel 510 using a tunnel dilator (not shown) such as the one described in U.S. Pat. No. 5,944,732 to Raulerson, et al. Next, the proximal end 304 of the tunneler 300 is initially inserted under a distal end 512 of the skin, proximate to the incision site where the catheter 500 has been inserted into the patient, and the tunneler 300 is drawn under the skin of the patient to form the tunnel 510, or through the tunnel 510 in the instance where a tunneler dilator (not shown) was used to form the tunnel 510. The physician draws the proximal end 304 of the tunneler 300 through the skin and exits the body of the patient out the proximal end 514 of the tunnel 510. If the tunneler 300 has a sharp tip 308, the initial incision that forms the proximal end 514 of the tunnel 510 may be omitted, and the tunneler 300 may be used to puncture the skin after tunneling, forming the proximal end 514 of the tunnel 510 at this time.

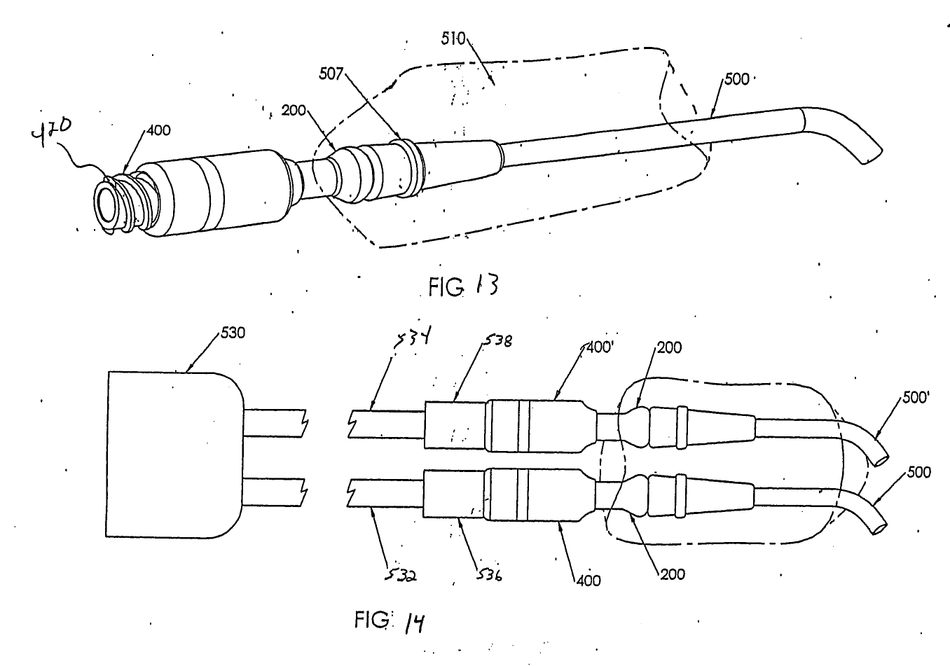

The assembly 100, comprising at this stage the catheter 500, the adapter 200 and the tunneler 300, is drawn through the tunnel 510 preferably at least until the adapter 200 is drawn partially through the tunnel 510, with the proximal end 204 of the adapter 200 being drawn from the proximal end 514 of the tunnel 510, but with the generally bulbous portion 208 of the adapter 200 remaining within the tunnel 510, preferably proximate to the proximal end 514 of the tunnel 510. The tunneler 300 is then removed from the adapter 200, and the port 400 is connected to the adapter 200. As shown in FIG. 12, the threaded connection 412 of the port 400 is threaded onto the threaded connection 214 of the adapter 200. Although FIG. 12 shows an adapter 200 having female threads and a port 400 having male threads, it will be known to those skilled in the art that the adapter 200 may have male threads and the port 400 may have corresponding female threads, or that some other method of connection, such as the compression sleeve 620 shown in FIG. 6, may be used to releasable connect the port 400 to the adapter 200. Referring back to FIG. 12, prior to threading the port 400 into the adapter 200, the inserting physician may snap a snap ring 260 onto the proximal end 204 of the adapter 200. This is done by snapping the distal ridge 266 of the snap ring 260 into the recess 230 of the adapter 200. The snap ring 260 will then engage the recess 430 in the distal portion 402 of the port when the port 400 is fully threaded into the adapter 200. While FIG. 12 shows an embodiment where the snap ring 260 is snapped onto the adapter 200 prior to releasably connecting the adapter 200 to the port 400, those skilled in the art will know that the snap ring 260 may also be snapped on to the port 400 prior to releasably connecting the adapter 200 to the port 400. Those skilled in the art will know that in an embodiment (not shown) wherein the port has female threads and the adapter has corresponding male threads, the snap ring 260 is preferably snapped onto the port prior to releasably connecting the port to the adapter. The port 400, having been connected to the adapter 200, is shown in FIG. 13. The luer connector 420 may be capped off and, if the catheter 500 has not already been inserted into the patient, the distal end of the catheter 500 may be inserted into the patient according to known techniques.

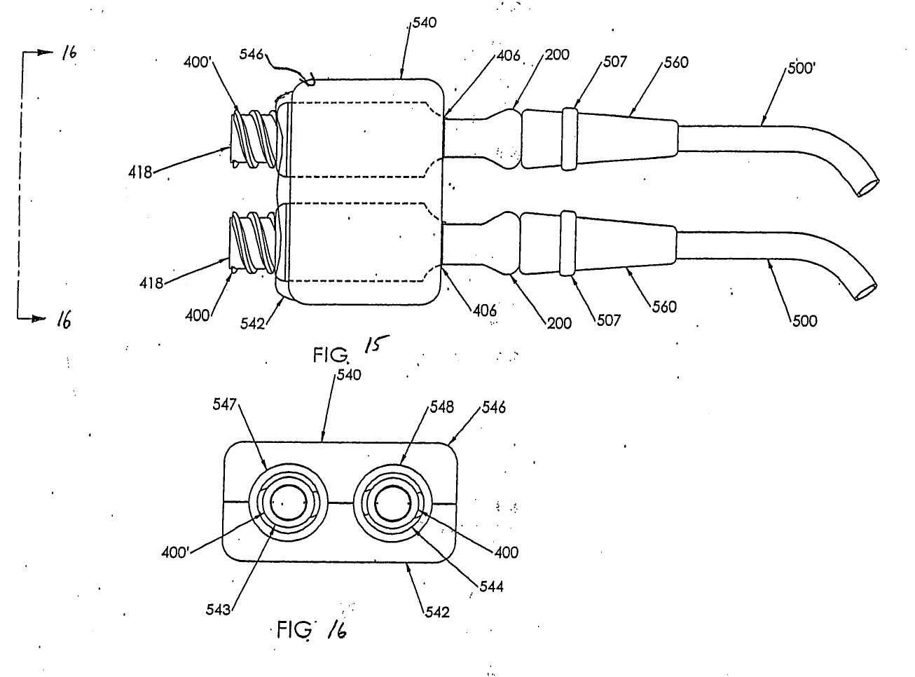

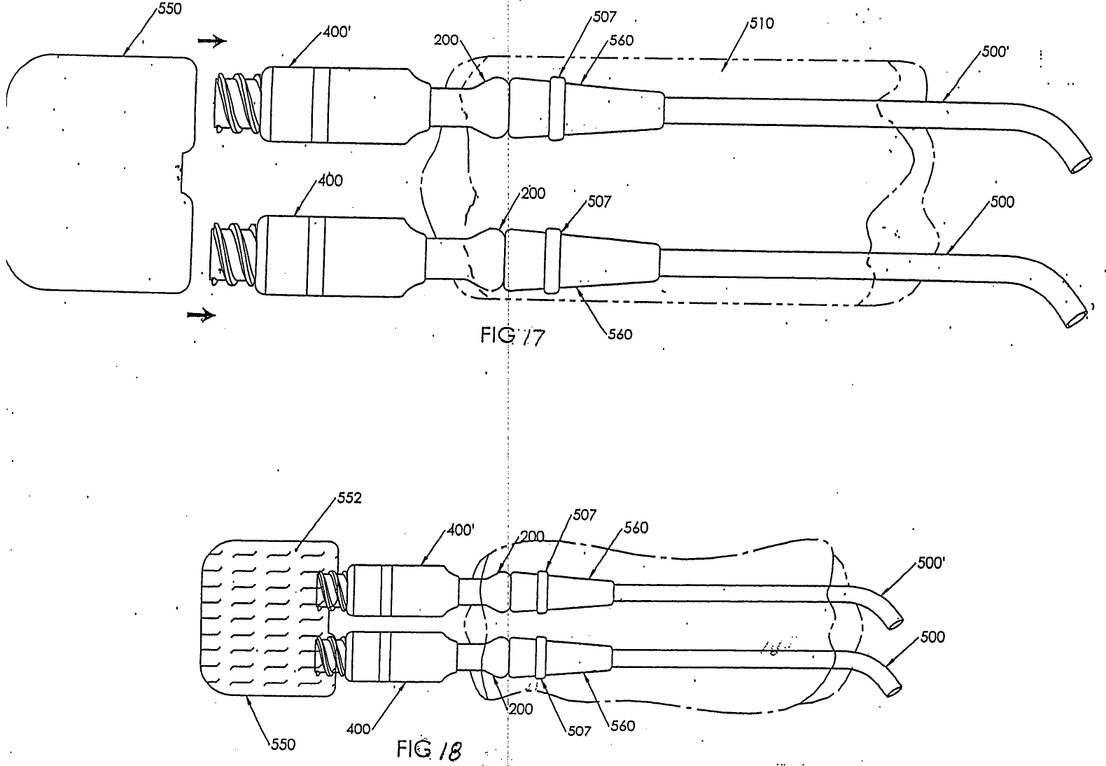

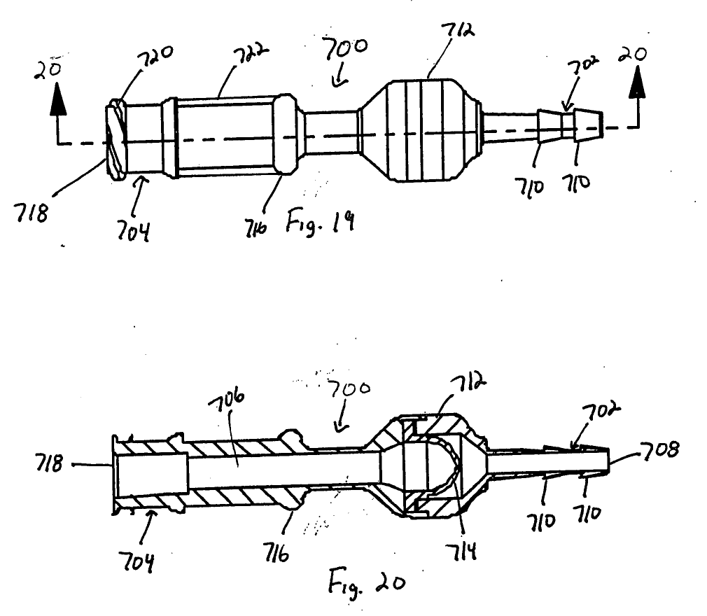

Typically, a hemodialysis catheterization arrangement in a patient consists of two catheters 500, 500′, shown in FIG. 14, with the catheter 500 adapted to withdraw blood from the patient for processing in a hemodialysis machine 530 and the catheter 500′ adapted to return the blood to the patient after the blood is processed in the hemodialysis machine 530. The catheter 500′ is inserted through the subcutaneous tunnel 510 in the same manner as described above with respect to the catheter 500. Dialysis machine tubes 532, 534 connect the ports 400, 400′, respectively, to the dialysis machine 530. Each tube 532, 534 includes a luer connector 536, 538, respectively, for connection to the luer connector 420 on the ports 400, 400′, respectively.

Preferably, the port 400 that is connected to the catheter 500 includes the valve 424 disposed within the port 400 to facilitate fluid flow in a first direction from the catheter 500 and the adapter 200, through the port 400 and to the dialysis machine 530, and to restrict fluid flow in a second direction from the dialysis machine 530, through the port 400 and to the catheter 500. The port 400′ is similar to the port 400, but the valve 424 is disposed within the port 400′ to facilitate fluid flow in the second direction from the dialysis machine 530, through the port 400′ and to the catheter 500′, and to restrict fluid flow in the first direction from the catheter and the adapter 200, through the port 400′ and to the dialysis machine 530. Alternatively, in the embodiment of the adapter shown in FIG. 3, the adapter includes the valve 272 and a corresponding port (not shown) has only a longitudinal passageway. The function of the valve 272 in the adapter 270 is substantially similar to the valve 424 in the port 400 described above.

To identify whether the port is the port 400 with the valve 424 disposed within the port 400 to facilitate flow in the first direction, or whether the port is the port 400′ with the valve disposed within the port 400′ to facilitate flow in the second direction, the ports 400, 400′ may be coded, such as with a color code, to distinguish between the port 400 and the port 400′. A like or similar code is also preferably present on each of the female luer connectors 536, 538 on the hemodialysis machine 530 to correspond to the port 400, 400′ to which each of the female luer connectors 536, 538 is to be connected during hemodialysis.

Optionally, as shown in FIGS. 15 and 16, the ports 400, 400′ may be inserted into a retainer 540. The retainer 540 includes a bottom portion 542 that may be connected to the patient, such as by suturing, or an adhesive. Alternatively, the bottom portion 542 may freely rest against the patient's skin. The bottom portion 542 includes a pair of generally semi-circularly shaped channels 543, 544 into which at least a portion of each of the ports 400, 400′ is inserted. As seen in FIG. 15, the distal end 406 and the proximal end 418 of each port 400, 400′ may extend beyond the retainer 540, with a portion of each port 400, 400′ inserted into the channels 543, 544, respectively.

Referring now to FIG. 16, the retainer 540 further includes a top portion 546 that includes a pair of generally semi-circularly shaped channels 547, 548 that are aligned with the channels 543, 544 of the bottom portion 542 of the retainer 540 when the top portion 546 is disposed over the bottom portion 542 for receiving the portion of each port 400, 400′ that is inserted into the channels 543, 544 in the bottom portion 542. The top portion 546 releasably connects to the bottom portion 542, such as by a snap-fit, or by some other, suitable, releasable connection.

Referring back to FIGS. 15 and 16, between dialysis treatments, the luer connectors 536, 538 are disconnected from the luer connectors 420 on the ports 400, 400′. After disconnecting, the catheters 500, 500′ and the ports 400, 400′ must be treated to reduce or prevent blood clotting and/or infection. To achieve this goal, each catheter 500, 500′ is primed with a locking solution consisting of a known antithrombotic and/or antiseptic, as is well known in the art. A syringe (not shown) filled with the locking solution is connected to the luer connector 420 on the port 400 and injected into the port 400. Alternatively, a medicine pouch (not shown) filled with the abovementioned locking solution may be connected to the luer connector 420 on the port 400 and squeezed to inject the locking solution into the port 400. Although the valve 424 is inserted into the port 400 to restrict flow in the direction from the proximal end 418 to the distal end 406, the force of the locking solution imparted by the syringe or medicine pouch is sufficient to overcome the restriction of the valve 424 and transmit the locking solution into the catheter 500. After filling the catheter 500 with the locking solution, the syringe or pouch is removed from the port 400. Similarly, a syringe or pouch (not shown) filled with the locking solution is connected to the luer connector 420 on the port 400′ and injected into the port 400′ to prime the catheter 500′. Since the valve 424 is disposed within the port 400′ to facilitate flow from the proximal end 418 to the distal end 406, the locking solution is easily transmitted past the valve 424 to the catheter 500′ for priming. After priming, the syringe or pouch is removed from the luer connector 420 on the port 400′.

After each of the catheters 500, 500′ are primed, a locking device 550, shown in FIG. 17, is disposed over the luer connectors 420 on each of the ports 400, 400′. As shown in FIG. 18, the locking device 550 includes locking solution 552 disposed therein. FIGS. 17 and 18 are shown without the optional retainer 540, for clarity. The luer connectors 420 are immersed in the locking solution 552. The locking solution 552 ensures that the luer connectors 420 remain generally patent and bacteria-free between hemodialysis treatments.

While a single locking device 550 that encompasses both ports 400, 400′ is shown, those skilled in the art will recognize that two independent locking devices (not shown), one for each of the ports 400, 400′, can be used. Optionally, the locking device 550 may be a flexible container, so that, between dialysis treatments, the patient, as directed by his/her physician, may be able to squeeze the locking device 550 to force additional priming solution into the catheters 500, 500′ to replace any of the initial priming solution that may have leaked from the catheters 500, 500′.

Referring now to FIGS. 19 and 20, there is an embodiment of the port assembly 700 wherein the port assembly 700 is connectable to a catheter and tunneler. The port assembly 700 has a generally tubular shape with a generally circular cross-section. The port assembly 700 comprises a distal portion 702, a proximal portion 704 and a longitudinal passageway 706 extending therethrough. A distal tip 708 is located at the distal portion 702 and is constructed to frictionally engage a catheter. The distal portion 702 has barbs 710 to increase the friction between the distal tip 708 and the catheter. The port assembly 700 also has a generally bulbous portion 712 located between the proximal portion 704 and the distal portion 702. The bulbous portion 712 has a diameter larger than the rest of the port assembly 700. Preferably, a valve 714 is located along the longitudinal passageway 706 within the bulbous portion 712. Preferably, the valve 714 allows fluid flow easily in a first direction but restricts fluid flow in a second direction. Those skilled in the art will recognize that the valve 714 is bidirectional. While the valve 714 is shown in FIG. 20 to allow fluid flow in a proximal to distal direction, the valve 714 may be disposed within the in the port assembly 700 to allow flow in a distal to proximal direction. It should be known that the valve 714 is bidirectional. The valve 714 allows flow in both directions but restricts flow in a non-preferred direction. This is particularly useful in applications such as hemodialysis wherein two separate catheters, or a multiple catheter assembly, utilizes two separate port assemblies 700 each with a valve 714 disposed to allow flow in opposite directions. A preferred valve is a bidirectional valve or a duckbill valve, although those skilled in the art will recognize that other types of valves may be used. Alternatively, the valve 714 may restrict fluid flow in both directions. Preferably, during manufacture of the port assembly 700, the valve 714 is inserted into the longitudinal passageway 706 prior to connecting the distal portion 702 and the proximal portion 704.

A gripping nub 716 is located proximally of the bulbous portion 712. Preferably, the gripping nub 716 has a diameter that is larger that the diameter of the port assembly 700, but smaller than the bulbous portion 712. Optionally, there may be a silicone ring (not shown) or ingrowth cuff (not shown) located about the exterior surface of the port assembly 700, such as the silicone ring 240 and ingrowth cuff 242 shown in FIG. 2. Referring back to FIGS. 19 and 20, a proximal tip 718 is located at the proximal end of the port assembly 700. Preferably, male threads 720 for a luer fitting (not shown) are located at the proximal tip 718. The threads 720 are constructed to be threadably connected to the tunneler 350 of FIGS. 4 and 5. Although FIGS. 19 and 20 show a port assembly 700 having male threads, those skilled in the art will recognize that female threads or any other connection method that will releasable connect the port assembly to the tunneler 350 may be used. Optionally, a silicone ring 722 may be located proximally of the retaining nub 716. The silicone ring 722 may be coded, preferably with a color code to denote which way flow is intended to pass through the port assembly 700.

The port assembly 700 is preferably used with the tunnelers 350, 600 shown in FIGS. 4 through 6. Referring now to FIGS. 20 and 5, when the port assembly 700 is threadably connected to the tunneler 350, the threads 720 of the port assembly 700 engage the threads 388 of the connector piece 380. When the threads 388, 720 are tightened together, the tapered sides 372 of the retaining nub 370 frictionally engage the surface of the longitudinal passageway 706 at the proximal tip 718.

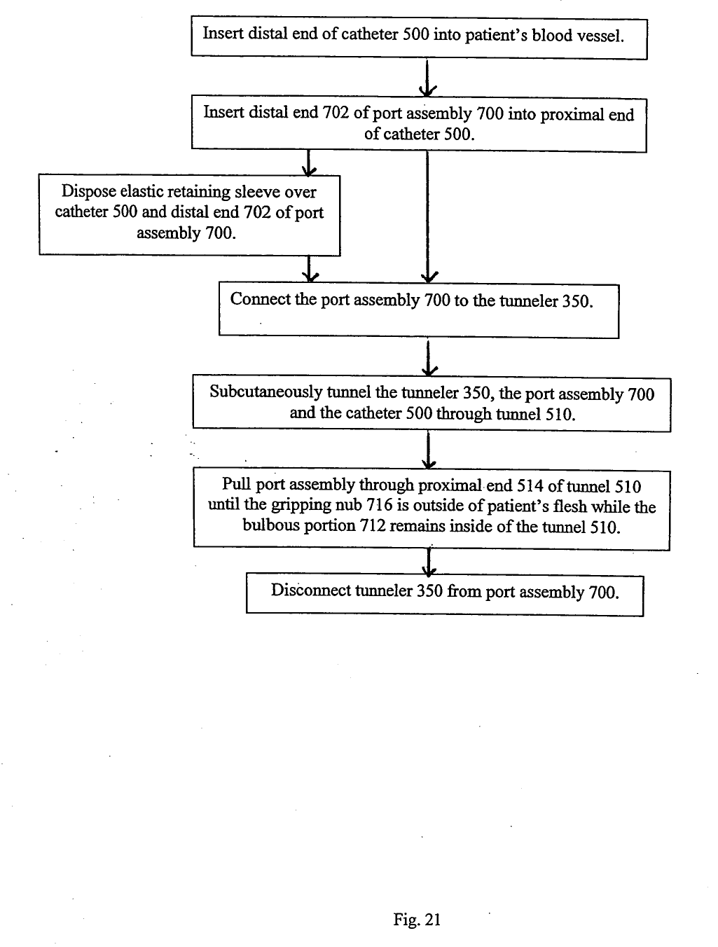

Referring now to the flow chart of FIG. 21, a method of subcutaneously tunneling the port assembly 700 of FIGS. 19 and 20 is shown. To use the port assembly 600, distal ends (not shown) of the catheters are surgically inserted into a patient's blood vessel according to known techniques. Optionally, the distal ends of the catheters may be inserted into the patient after the port assembly 700 is connected to the catheter. The distal portion 702 of the port assembly 700 is inserted into the proximal end of a lumen of a catheter. The distal portion 702 of the port assembly 700 is inserted sufficiently into the lumen such that the catheter extends over the barbs 710. The catheter engages the distal portion 702 of the port assembly 700 in a leak proof fit. Optionally, an elastic retaining sleeve (not shown) is disposed over the catheter 500 and the distal portion 702 of the port assembly 700 to further compress the catheter onto the distal portion 702 of the adapter 700. FIG. 9 shows an example of the retaining sleeve 506. The retaining sleeve 506 is preferably constructed from silicone or some other suitable biocompatible material known to those skilled in the art. Also optionally, a fabric ingrowth cuff 507 may be disposed about at least a portion of the exterior of the retaining sleeve to facilitate securing the catheter to the patient after the catheter is tunneled. As seen in FIG. 9, the retaining sleeve 506 is preferably tapered, although those skilled in the art will recognize that the retaining sleeve 506 need not be tapered. Referring back to the flowchart of FIG. 20, once the distal portion 702 of the port assembly 700 is inserted into the catheter, the port assembly 700 is then threadably connected to the tunneler 350. Next, the port assembly 700, with the catheter and tunneler 350, are subcutaneously tunneled in accordance with the method described above with respect to the adapter 200 and the tunneler 300. The port assembly 700 is then pulled through the tunnel so that the gripping nub 716 is outside of the patient's flesh and the bulbous portion 712 is left under the patients flesh. The tunneler 350 is then disconnected from the port assembly 700.

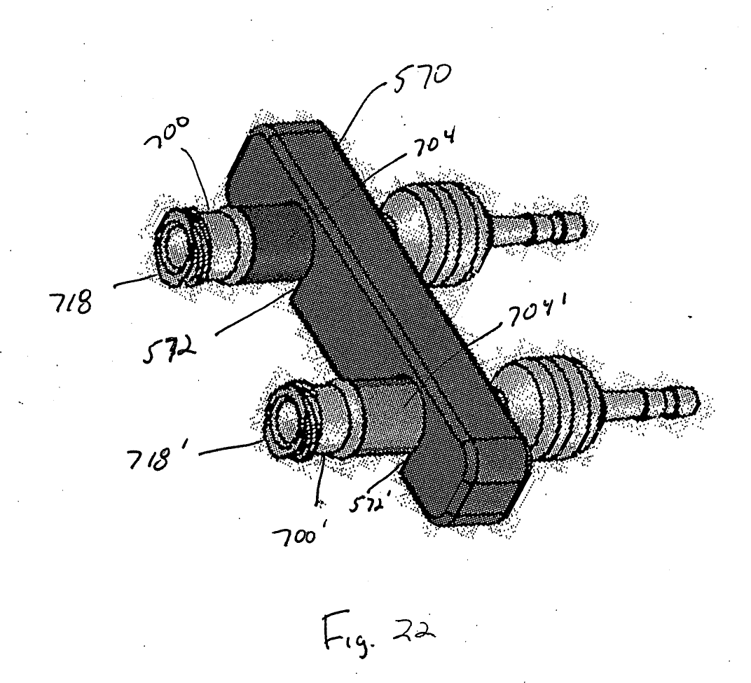

As shown in FIG. 22 the tunneled port assembly may be inserted into a snap retainer 570. The snap retainer 570 is preferably constructed to hold the proximal tips 718, 718′ of the port assemblies 700, 700′ in close proximity and parallel to each other. The snap retainer 570 is designed with one or more retaining grooves 572, 572′ constructed to snugly fit around the proximal portion 704, 704′ of the port assembly 700, 700′. Those skilled in the art will recognize that use of the snap retainer 570 is not limited to use with the port assembly 700 and that the snap retainer 570 may be used with all of the assemblies described herein. Likewise, those skilled in the art will recognize that the port assembly 600 may be used with all of the retainers described herein. While FIG. 22 shows a snap retainer 570 disposed over the ports, those skilled in the art will recognize that the snap retainer 570 may be placed under the ports 700, 700′. Those skilled in the art will also recognize that the snap retainer 570 may be constructed to angle the ports 700, 700′away from the patient's body to allow for easy access to the ports 700, 700′.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A catheter port assembly comprising:
a body having an outer surface, a proximal end, a distal end and a passageway extending therethrough between the proximal end and the distal end:
wherein the body is releasably connectable to one of a port and a tunneler; and
wherein a valve is disposed within at least one of the body and the port.

2. The catheter port assembly according to claim 1, wherein the port is integral with the body.

3. The catheter port assembly according to claim 1, wherein the port is releasably connected to the body.

4. The catheter port assembly according to claim 3, further comprising a snap ring releasably lockable around the body and the port.

5. The catheter port assembly according to claim 1, wherein the tunneler is releasably connectable to the port.

6. The catheter port assembly according to claim 1, wherein only one of the tunneler and the catheter port are connectable to the proximal end of the body at a particular time.

7. The catheter port assembly according to claim 1, wherein the valve allows fluid flow in a first direction, but restricts fluid flow in a second direction.

8. The catheter port assembly according to claim 1, wherein the valve restricts fluid flow in all directions.

9. The catheter port assembly according to claim 1, wherein the catheter port comprises a code.

10. The catheter port assembly according to claim 9, wherein the code is a color code.

11. The catheter port assembly according to claim 1, wherein the distal end of the body is generally tapered.

12. The catheter port assembly according to claim 1, wherein the distal end of the body comprises at least one raised portion.

13. The catheter port assembly according to claim 12, wherein the raised portion is a barb.

14. The catheter port assembly according to claim 1, wherein the proximal end of the body includes a threaded connection.

15. The catheter port assembly according to claim 1, further comprising a retainer releasably connected to the body.

16. The catheter port assembly according to claim 1, further comprising a locking device releasably connected to a proximal end of the body.

17. The catheter port assembly according to claim 16, wherein the locking device further comprises a catheter locking solution disposed therein.

18. The catheter port assembly according to claim 1, further comprising an O-ring disposed inside the passageway of the body proximal to the distal end of the body.

19. The catheter port assembly according to claim 1, further comprising an O-ring disposed inside a longitudinal passageway of the catheter port proximal to a distal end of the catheter port.

20. The catheter port assembly according to claim 1, wherein a ring is disposed around the outer surface of the body.

21. The catheter port assembly according to claim 20, wherein a cuff is fixedly attached to the ring.

22. The catheter port assembly according to claim 1, wherein the tunneler is constructed of two or more pieces.

23. The catheter port assembly according to claim 22, wherein the tunneler comprises an elongated shaft and a connector piece.

24. The catheter port assembly according to claim 23, wherein the connector piece is releasably connectable to the body.

25. The catheter port assembly according to claim 23, wherein the connector piece rotates freely about the elongated shaft.

26. A catheter port assembly comprising:
a body having an outer surface, a proximal end, a distal end, a passageway extending therethrough between the proximal end and the distal end, a valve disposed along the longitudinal passageway between the distal end and the proximal end and a tunneler releasably connectable to the body.

27. The catheter port assembly according to claim 26, wherein the valve allows fluid flow in a first direction, but restricts fluid flow in a second direction.

28. The catheter port assembly according to claim 26, wherein the valve restricts fluid flow in all directions.

29. The catheter port assembly according to claim 26, wherein the catheter port comprises a code.

30. The catheter port assembly according to claim 29, wherein the code is a color code.

31. The catheter port assembly according to claim 26, wherein the distal end of the body is generally tapered.

32. The catheter port assembly according to claim 26, wherein the distal end of the body comprises at least one raised portion.

33. The catheter port assembly according to claim 32, wherein the raised portion is a barb.

34. The catheter port assembly according to claim 26, wherein the proximal end of the body includes a threaded connection.

35. The catheter port assembly according to claim 26, further comprising a retainer releasably connected to the body.

36. The catheter port assembly according to claim 26, further comprising a locking device releasably connected to a proximal end of the body.

37. The catheter port assembly according to claim 36, wherein the locking device further comprises a catheter locking solution disposed therein.

38. The catheter port assembly according to claim 26, wherein a ring is disposed around the outer surface of the adapter.

39. The catheter port assembly according to claim 38, wherein a cuff is fixedly attached to the ring.

40. The catheter port assembly according to claim 26, wherein the tunneler is constructed of two or more pieces.

41. The catheter port assembly according to claim 40, wherein the tunneler comprises an elongated shaft and a connector piece.

42. The catheter port assembly according to claim 41, wherein the connector piece is releasably connectable to the body.

43. The catheter port assembly according to claim 41, wherein the connector piece rotates freely about the elongated shaft.

44. A method of subcutaneously securing a catheter to a patient comprising:
a) providing a catheter port assembly comprising a body having an outer surface, a proximal end, a distal end and a passageway extending therethrough between the proximal end and the distal end, wherein the body is releasably connectable to one of a port and a tunneler; and wherein a valve is disposed within at least one of the body and the port;
b) releasably connecting the tunneler to the body;
c) using the tunneler to form a subcutaneous tunnel in a patient;
d) advancing the tunneler and at least a portion of the assembly through the tunnel and out of the tunnel;
e) removing the tunneler from the body.

45. The method according to claim 44, wherein removing the tunneler from the adapter comprises unthreading the tunneler from the body.

46. The method according to claim 44, further comprising releasably connecting a distal end of a catheter port to the proximal end of the body.

47. The method according to claim 46 further comprising, prior to connecting the distal end of a catheter port to the proximal end of the body, attaching a snap ring to the distal end of the catheter port, wherein the snap ring is sized to snap into a recess located on the proximal portion of the body.

48. The method according to claim 46 further comprising attaching a snap ring to the proximal portion of the catheter body, wherein the snap ring is sized to snap into a recess located on the distal end of the catheter port.

49. The method according to claim 46, wherein releasably connecting the distal end of the catheter port to the proximal end of the body comprises threadably connecting the distal end of the catheter port to the proximal end of the body.

50. The method according to claim 46, wherein releasably connecting the distal end of the catheter port to the proximal end of the body comprises stretching a compression sleeve around the proximal end of the body and the distal end of the catheter port.

51. The method according to claim 44, wherein the catheter port comprises a valve disposed therein.

52. The method according to claim 44, further comprising, after removing the tunneler from the body, connecting a proximal end of the catheter port assembly to a locking device.

53. The method according to claim 52, wherein connecting the proximal end of the catheter port to the locking device comprises immersing the proximal end of the catheter port in at least one of an antithrombotic and an antiseptic solution.

54. The method according to claim 44, further comprising, after removing the tunneler from the body, connecting a proximal end of the catheter port assembly to a medical device.

55. The method according to claim 54, wherein the medical device comprises a hemodialysis machine.

56. The method according to claim 44, further comprising, after removing the tunneler from the body, connecting a proximal end of the catheter port assembly to a syringe.

57. The method according to claim 44, further comprising, after removing the tunneler from the body, connecting a proximal end of the catheter port assembly to a medicine pouch.
